(19) United States
(12) Patent Application Publication
(10) Pub. No.: US 2023/0277435 A1
(43) Pub. Date: Sep. 7, 2023

(54) COSMETIC MAKE-UP COMPOSITION FOR THE LIPS

(71) Applicant: L V M H RECHERCHE, Saint-Jean De Brave (FR)

(72) Inventors: Gaelle Toressani, Saint-Jean De Brave (FR); Marine Fabris, Saint-Jean De Brave (FR)

(21) Appl. No.: 18/005,792

(22) PCT Filed: Jul. 16, 2021
(86) PCT No.: PCT/FR2021/051332

§ 371 Date: Jan. 17, 2023

(30) Foreign Application Priority Data
Jul. 17, 2020 (FR) F2007523

(51) Int. Cl. A61K 8/891; A61K 8/92; A61Q 1/06

(52) U.S. Cl. CPC A61K 8/891; A61K 8/922; A61Q 1/06; A61K 2800/31; A61K 2800/43

(57) ABSTRACT

A cosmetic composition, which is preferably anhydrous, includes, in a physiologically acceptable medium, at least: an organopolysiloxane elastomer conveyed in a first oil, a nonpolar oil distinct from the first oil, a polar wax, and pigments in a total content of greater than or equal to 10% by weight relative to the total weight of the composition.

### CROSS REFERENCE TO RELATED APPLICATIONS

This application is a U.S. National Stage of International Patent Application No. PCT/FR2021/051332 filed on Jul. 16, 2021, which claims the benefit of priority of French Patent Application No. 2007523 filed Jul. 17, 2020, the respective disclosures of which are each incorporated herein by reference in their entireties.

### FIELD OF THE INVENTION

The present invention relates to a make-up composition for the lips.

### PRIOR ART

Lip care and make-up products intended to embellish, protect and/or colour the lips are widespread. In particular, make-up products for the lips are known in liquid, semi-fluid or solid (stick) form for a glossy deposit or matt deposit on the lips and having good colour holding properties. Volatile oils, fillers and pigments are generally used to provide the mattness and coverage, but it is known that increasing the total content of pigments, in particular beyond 8% to 10% by weight of pigments, can affect the stability of the composition and the comfort of the application; the film deposit can have an inhomogeneous and desiccating appearance on the lips.

There is therefore still a need to develop new lip make-up products, in particular solid products, with a large total content of pigments and which are stable, in order to obtain a homogeneous and comfortable deposit, having good coverage that is matt and is not desiccating on the lips. Consumers also desire soft properties on application, with a “velvety” effect.

The present invention responds to these needs. Indeed, the applicant has developed a composition comprising a total content of pigments greater than or equal to 10% by weight of the composition and an organopolysiloxane elastomer conveyed in a first oil, in an oily phase containing at least one non-polar oil and a polar wax. Unexpectedly, the inventors have obtained a composition having very good stability despite the high content of pigments and the incompatibility of the non-polar oil with the polar wax, with the desired properties of coverage, mattness, comfort and hydration on application, with a velvety deposit.

### DISCLOSURE OF THE INVENTION

The invention relates, according to a first aspect, to a cosmetic composition comprising, in a physiologically acceptable medium, at least:
an organopolysiloxane elastomer conveyed in a first oil,
a non-polar oil, distinct from the first oil,
a polar wax, and
pigments with a total content greater than or equal to 10% by weight relative to the total weight of the composition.

The invention also relates to a method for cosmetic make-up of the lips, comprising the application, on the lips, of at least one layer of a composition according to the invention.

### DETAILED DESCRIPTION OF THE INVENTION

A first object of the invention is thus a cosmetic composition comprising, in a physiologically acceptable medium, at least:
an organopolysiloxane elastomer conveyed in a first oil,
a non-polar oil, distinct from the first oil,
a polar wax, and
pigments with a total content greater than or equal to 10% by weight relative to the total weight of the composition.

According to a particular and preferred embodiment, the composition of the invention is an anhydrous composition.

The term “anhydrous” shall mean, in particular, that water is preferably not deliberately added to the compositions but may be present in trace amounts in the various compounds used in the compositions. In particular, the composition according to the invention comprises less than 4% by weight water, preferably less than 3%, preferably less than 2%, more preferably less than 1%, yet more preferably less than 0.5% by weight water, relative to the total weight of said composition, or is even totally devoid of water.

The composition of the invention is generally a solid composition.

The term “solid” shall mean a composition having, at a temperature of 20° C. and atmospheric pressure (760 mm Hg), a hardness greater than 30 Nm¹⁻, preferably greater than 40 Nm⁻¹.

The hardness can be measured at 20° C. by the so-called “butter wire” method, which consists of transversely cutting a baton of preferably cylindrically symmetric product, using a rigid metal wire of diameter 250 μm by moving the wire relative to the stick at a speed of 60 mm/min. The hardness of the samples is expressed in grams-force (gf) and can be measured by means of a texture analyser such as a TAXT Plus Texture Analyser. Preferably, the value obtained will be between 140 and 190.

The cosmetic composition of the invention comprises a fatty or oily phase.

The term “oily phase” shall mean an oil or a mixture of mutually miscible oils.

Within the meaning of the invention, the term “oil” shall mean a fatty body that is not soluble in water and is liquid at 25° C. and atmospheric pressure.

The composition of the invention comprises at least one first oil, which serves to convey the organopolysiloxane elastomer and a second oil distinct from the first oil. These oils are described below.

### Organopolysiloxane Elastomer Conveyed in a First Oil

Within the meaning of the invention, the term “conveyed” shall mean that the elastomer is transported in the composition in a pre-dispersed form in at least a first oil, in particular in the form of a homogeneous mixture of elastomer particles dispersed in the first oil, that is stable for at least 24 hours at 20° C. Preferably, this elastomer is in the form of a gel in at least a first oil.

The term “organopolysiloxane elastomer” or “silicone elastomer” shall mean a flexible, deformable organopolysiloxane having viscoelastic properties and, in particular, the consistency of a sponge or a flexible sphere. Its modulus of elasticity is such that this material resists deformation and has a limited capacity for extension and contraction. This material is capable of returning to its original shape following stretching.

More particularly, it is a cross-linked silicone elastomer.

The elastomer can be chosen among non-emulsifying or emulsifying elastomers. Preferably, it is a non-emulsifying elastomer.

The term “non-emulsifying” defines organopolysiloxane elastomers which do not contain a hydrophilic chain, and in particular not containing polyoxyalkylene units (in particular polyoxyethylene or polyoxypropylene units), nor polyglyceryl units.

Hence, the organopolysiloxane elastomer can be obtained by:
a cross-linking addition reaction of diorganopolysiloxane containing at least one hydrogen bonded to silicon and diorganopolysiloxane having ethylenically unsaturated groups bonded to silicon, in particular in the presence of a platinum catalyst;
or by a cross-linking, hydrogenation, condensation reaction between a diorganopolysiloxane with hydroxyl end groups and a diorganopolysiloxane containing at least one hydrogen bonded to silicon, in particular in the presence of an organotin;
or a cross-linking condensation reaction of a diorganopolysiloxane with hydroxyl end groups and a hydrolysable organopolysiloxane;
or by thermal cross-linking of organopolysiloxane in particular in the presence of an organic peroxide catalyst;
or by cross-linking organopolysiloxane by high-energy radiation such as gamma rays, ultra-violet rays or an electron beam.
Preferably, the organopolysiloxane elastomer is obtained by a cross-linking addition reaction (A) of diorganopolysiloxane containing at least two hydrogens, each bonded to a silicon, and (B) diorganopolysiloxane having at least two ethylenically unsaturated groups bonded to silicon, in particular in the presence (C) of a platinum catalyst.
In particular, the organopolysiloxane elastomer can be obtained by reaction of dimethylpolysiloxane with dimethylvinylsiloxy end groups and methylhydrogen polysiloxane with trimethylsiloxy end groups, in the presence of a platinum catalyst.
Compound (A) is the base reagent for forming organopolysiloxane elastomer and the cross-linking takes place by addition reaction of compound (A) with compound (B) in the presence of the catalyst (C).
Compound (A) is, in particular, an organopolysiloxane having at least two hydrogen atoms bonded to distinct silicon atoms in each molecule. Compound (A) may have any molecular structure, in particular a linear chain or branched chain structure or a ring structure.
Compound (A) can have a viscosity at 25° C. ranging from 1 to 50,000 centistokes, in particular in order to be easily miscible with compound (B). The organic groups bonded to the silicon atoms of compound (A) may be alkyl groups such as methyl, ethyl, propyl, butyl, octyl; substituted alkyl groups such as 2-phenylethyl, 2-phenylpropyl, 3,3,3-trifluoropropyl; aryl groups such as phenyl, tolyl, xylyl; substituted aryl groups such as phenylethyl; and substituted monovalent hydrocarbon groups such as an epoxy group, a carboxylic ester group or a mercapto group.
Compound (A) can thus be chosen from methylhydrogenpolysiloxanes with trimethylsiloxy end groups, dimethylsiloxane-methylhydrogensiloxane copolymers with trimethylsiloxy end groups, and cyclic dimethylsiloxane-methylhydrogensiloxane copolymers. Compound (B) is advantageously a diorganopolysiloxane having at least two lower alkenyl groups (for example C2-C4); the lower alkenyl group may be chosen from vinyl, allyl and propenyl groups. These lower alkenyl groups can be located in any position on the organopolysiloxane molecule but preferably not located at the ends of the organopolysiloxane molecule. Organopolysiloxane (B) can have a branched chain, linear chain, ring or network structure but the linear chain structure is preferred. Compound (B) can have a viscosity ranging from the liquid state to the rubber state. Preferably, compound (B) has a viscosity of at least 100 centistokes at 25° C.
In addition to the above mentions alkenyl groups, the other organic groups bonded to the silicon atoms in compound (B) can be alkyl groups such as methyl, ethyl, propyl, butyl or octyl; substituted alkyl groups such as 2-phenylethyl, 2-phenylpropyl or 3,3,3-trifluoropropyl; aryl groups such as phenyl, tolyl or xylyl; substituted aryl groups such as phenylethyl; and substituted monovalent hydrocarbon groups such as an epoxy group, a carboxylic ester group, or a mercapto group.
The organopolysiloxanes (B) can be chosen from methylvinylpolysiloxanes, methylvinylsiloxane-dimethylsiloxane copolymers, dimethylpolysiloxanes with dimethylvinylsiloxy end groups, dimethylsiloxane-methylphenylsiloxane copolymers with dimethylvinylsiloxy end groups, dimethylsiloxane-diphenylsiloxanemetliylvinylsiloxane copolymers with dimethylvinylsiloxy end groups, dimethylsiloxanemethylvinylsiloxane copolymers with trirnethylsiloxy end groups, dimethylsiloxanemethylphenylsiloxane-methylvinylsiloxane copolymers with trirnethylsiloxy end groups, methyl(3,3,3-trifluoropropyl)polysiloxane with dimethylvinylsiloxy end groups, and dimethylsiloxane-methyl(3,3,3-trifluoropropyl)siloxane copolymers with dimethylvinylsiloxy end groups. In particular, the organopolysiloxane elastomer can be obtained by reaction of dimethylpropylisoxane with dimethylvinylsiloxy end groups and methylhydrogenpolysiloxane with trimethylslioxy end groups, in the presence of a platinum catalyst. According to another alternative, compound (B) can be an unsaturated hydrocarbon compound having at least two lower alkenyl groups (for example with C2-C4); the lower alkenyl group can be chosen from the vinyl, allyl and propenyl groups. These lower alkenyl groups can be located in any position on the molecule but are preferably located at the ends. Examples include hexadiene, and in particular the 1,5-hexadiene.
Advantageously, the sum of the number of ethylenic groups per molecule of compound (B) and the number of hydrogen atoms bonded to silicon atoms per molecule of compound (A) is at least 5.
It is advantageous that compound (A) is added in a quantity such that the molecular ratio between the total quantity of hydrogen atoms bonded to silicon atoms in compound (A) and the total quantity of all the ethylenically unsaturated groups in compound (B) is in the range from 1.5/1 to 20/1.
Compound (C) is the cross-linking reaction catalyst and is, in particular, chloroplatinic acid, chloroplatinic acid-olefin complexes, chloroplatinic acid-alkenylsiloxane complexes, chloroplatinic acid-diketone complexes, platinum black and the platinum on the support. The catalyst (C) is preferably added at from 0.1 to 1000 parts by weight, better from 1 to 100 parts by weight, as clean platinum metal for 1000 parts by weight of the total quantity of compounds (A) and (B).
Examples of non-emulsifying elastomers which can be used include those sold under the names DOWSIL Silicone Elastomer Blend such as that conveyed in a non-volatile oil sold under the name “DOWSIL, 9041” and “DOWSIL, EL-9241 DM” by Dow Chemical Company, as well as those conveyed in a volatile oil sold under the name “DOWSIL EL-8040 ID”, “DOWSIL EL-9140 DM”, “DOWSIL EL-9240 DM”, “DOWSIL EL-9048”, “DOWSIL 9040”, “DOWSIL 9045”, “DOWSIL EB-9586”, “DOWSIL 9546” by the Dow Chemical Company.
Mention can also be made to those sold under the names “KSG-15”.“KSG-1510”, “USG-1G3”, “USG-106”, “KSG-16”, “KSG-1610”, “KSG-18A”,“KSG-19”, “KSG-016F”, KSG-41 A”, “K SG -42 A”, “KSG-43”, “KSG-44” “KSG-042Z” “SG-045Z”, “KSG 048Z” by Shin Etsu. Other non-emulsifying elastomers conveyed in a non-volatile or weakly volatile oil are also sold under the names “Gransil DM-10”, “Gransil DMAM”, “Gransil DMG-20”, “Gransil DMG-6”, “Gransil PM” by Grant Industries; or those conveyed in a mixture of oils sold under the names “Gransil OHS-5”, “Gransil PS-5” by Grant industries; or those conveyed in a volatile oil sold under the names “GI CD-10”, “GI CD-11”. “Gransil DMG-” Gransil DMG-3”. “Gransil GCM-5”, “Gransil GTS”, “Gransil G VL”, “Gransil GVL-HV”, “Gransil IDS-5”, “Gransil MLB”, “Gransil PC-12”, “Gransil PC-12P”, “Gransil RPS”, “Gransil RPS-D6”, “GI CD-965”, “Gransil DM-5”, “Gransil DMCM-5”, “Gransil DMDM-25”, “Gransil DMDM-35”, “Gransil DMID”, “Gransil DMT3” “Gransil G AM” by Grant Industries.

In particular, the composition according to the invention comprises a content of organopolysiloxane elastomer or elastomers conveyed in a first oil, with a dry matter content (active ingredient) ranging from 1% to 10% by weight, preferably from 2% to 7% by weight, and in a yet more preferred manner from 3% to 6% by weight, relative to the total weight of the composition.

The term “first oil” shall mean the oil in which the organopolysiloxane elastomer is conveyed. This first oil is chosen from non-volatile oils and volatile oils, preferably non-volatile oils. It is, in particular, a silicone oil.

In the context of the invention, “silicone oil” shall mean an oil comprising at least one silicon atom, and in particular an Si—O group.

Examples include non-volatile polydimethylsiloxanes (PDMS), polydimethylsiloxanes including at least one C₂₋₂₄, alkyl or alkoxy group that is hanging and/or at the end of the silicone chain. Polydimethylsiloxanes include, in particular, heptamethyl hexyltrisiloxane, heptamethyloctyl trisiloxane, hexamethyl disiloxane, octamethyl trisiloxane, decamethyl tetrasiloxane, dodecamethyl pentasiloxane and the mixtures thereof. In particular, the first oil is chosen from linear polydimethylsiloxanes of viscosity between 5 cSt and 100 cSt (centistokes); or one of the mixtures thereof.

### Non-Polar Oil

The cosmetic composition according to the invention comprises at least one “second oil”, distinct from the first oil mentioned above. This second oil is chosen from non-volatile oils or volatile oils, or the mixture thereof, preferably non-volatile oils.

The second oil according to the invention is a “non-polar oil” otherwise referred to as an “apolar oil”.

Within the meaning of the invention, “non-polar oil” shall mean an oil for which the solubility parameter at 25° C., δa, is equal to 0 (J/cm³)^{1/2}. The definition and calculation of the HANSEN three-dimensional solubility parameters are described in the article by C. M. HANSEN: “The three dimensional solubility parameters” J. Paint Technol. 39, 105 (1967).

The non-polar oil may be a silicon oil or a hydrocarbon oil.

In particular, it will be a non-polar hydrocarbon oil.

The non-polar hydrocarbon oil can be chosen from linear or branched hydrocarbons of mineral or synthetic origin, such as: paraffin oil or the derivatives thereof, squalane, iso-icosane, naphthalene oil, polybutylenes, hydrogenated polyisobutylenes, decene/butene copolymers, polybutene/polyisobutene copolymers, polydecenes and hydrogenated polydecenes, and the mixtures thereof.

Preferably, the composition of the invention comprises at least one non-polar oil chosen from polybutylenes, hydrogenated polyisobutylenes, decene/butene copolymers, polybutene/polyisobutene copolymers, hydrogenated polydecenes and polydecenes, more preferably hydrogenated polydecenes.

The content of non-polar oil(s) according to the invention will generally range from 15% to 45%, in particular from 20% to 40%, by weight relative to the total weight of said composition.

The composition of the invention may further comprise other additional oils chosen from hydrocarbon oils, silicone oils and the mixtures thereof.

The total quantity of oils present in the composition according to the invention is generally between 40% and 70% by weight, relative to the total weight of the composition.

### Organopolysiloxane Elastomer Powder, Coated or not with Silicone Resin

The composition of the invention may further comprise an organopolysiloxane elastomer how distinct from the organopolysiloxane elastomer described above conveyed in an oil, said organopolysiloxane elastomer powder being coated or not coated with silicone resin. Advantageously, the composition can further comprise at least one silicone elastomer powder coated with silicone resin.

According to a particular embodiment, the composition according to the invention may comprise an organopolysiloxane elastomer powder that is not coated with silicone resin. It is possible to use as elastomers in the form of a powder, those sold under the names “DC9505”, “DC 9506” by Dow Corning and with name INCI Dimethicone/Vinyl Dimethicone Crosspolymer.

According to another particular and preferred embodiment, the composition according to the invention may comprise an organopolysiloxane elastomer powder coated with silicone resin, in particular silsesquioxane resin, as described for example in U.S. Pat. No. 5,538,793. Such elastomer powders are sold under the names “KSP-100”, “KSP-101”, “KSP-102”, “KSP-103”, “KSP-104”, “KSP-105” by Shin Etsu, and have INCI name “vinyl dimethicone/methicone silsesquioxane crosspolymer”.

Preferably, the organopolysiloxane elastomer powder coated with silicone resin is a compound with INCI name “vinyl dimethicone/methicone silsesquioxane crosspolymer”. Preferably, the composition according to the invention may further comprise an organopolysiloxane elastomer powder coated with silicone resin, in particular silsesquioxane resin, at a content ranging from 0.5% to 10% by weight, relative to the total weight of the composition, in particular from 1% to 5% by weight, preferably ranging from 2% to 4% by weight, relative to the total weight of the composition.

The composition may thus comprise, according to a particular embodiment of the invention, an organopolysiloxane elastomer conveyed in a non-volatile oil associated with at least one organopolysiloxane elastomer powder distinct from the organopolysiloxane elastomer described above conveyed in the oil, the organopolysiloxane elastomer powder being advantageously coated with a silicone resin.

The organopolysiloxane elastomer conveyed in the oil and the organopolysiloxane elastomer powder distinct from the organopolysiloxane elastomer described above and conveyed in the oil, can be present with a total dry matter content (active ingredient) ranging from 1% to 12%, in particular from 2% to 10%, and preferably from 3% to 8% by weight, relative to the total weight of said composition.

### Polar Wax

The composition according to the invention comprises at least one polar wax.

Within the meaning of the present invention, the term “wax” shall mean a solid compound at 25° C. which undergoes a reversible solid/liquid change of state and has a melting temperature greater than 30° C., preferably greater than 45° C.

In particular, the polar wax is chosen from the group comprising beeswax, camauba wax, candelilla wax, cotton wax, rice bran wax, sunflower wax, bay wax, Chinese insect wax, montan wax, lanolin and the acetylated, esterified, polyethoxylated alcohol derivatives thereof, kapok wax, sugar cane wax, hexyl laurate, jojoba wax, shellac wax, polyethoxylated cholesterol ether, synthetic beeswaxes marketed by Koster Keunen under the tradename Kester Wax K82H, or one of the mixtures thereof.

According to a particular embodiment, the composition of the invention comprises one or more polar waxes chosen from the group consisting of candelilla wax, beeswax, camauba wax, rice bran wax, sunflower wax, jojoba wax and the mixtures thereof, preferably candelilla wax.

According to a preferred embodiment, the composition of the invention comprises at least one candelilla wax as polar wax.

The polar wax(es) content in the composition of the invention will generally be from 0.5% to 10%, preferably from 1% to 5% by weight relative to the total weight of said composition.

The composition may further comprise a non-polar wax.

The term “non-polar wax” shall mean a hydrocarbon wax and/or a silicone wax. The term “non-polar hydrocarbon wax” shall mean a wax comprising only carbon and hydrogen atoms and not comprising heteroatoms such as oxygen, nitrogen, silicon or phosphorus. In particular, it is possible to use, as a non-polar wax, monocrystalline waxes, paraffins, ozokerite, polyethylene waxes and the mixtures thereof, preferably polyethylene waxes.

The term “non-polar silicone wax” shall mean a wax comprising a heteroatom of silicon.

Examples of non-polar silicone waxes include C20-24 alkyl dimethicone, marketed by Siltech under the name Silwax D2024; C24-28 alkyl dimethicone, marketed by Evonik Industries AG under the name Abil Wax, or one of the mixtures thereof.

The non-polar wax(es) content in the composition of the invention can range from 0.5 to 10%, preferably from 2 to 8% by weight relative to the total weight of said composition.

According to a particular embodiment, the composition of the invention will further comprise the polar wax, also a non-polar wax, preferably chosen from polyethylene waxes.

Hence, according to a particular embodiment, the composition of the invention comprises at least one candelilla wax and a polyethylene wax.

In particular, a composition according to the invention may comprise a total wax(es) content ranging from 2% to 15%, for example from 3% to 12% by weight, preferably from 5% to 10% by weight, relative to the total weight of the composition.

According to a particular embodiment, the total wax(es) content in the composition of the invention is greater than or equal to 5% by weight relative to the total weight of said composition.

### Pigments

The make-up composition of the invention further comprises pigments at a content greater than or equal to 10% by weight relative to the total weight of said composition. In particular, the pigments are present at a content ranging from 10% to 20% by weight, in particular from 11% to 18% by weight relative to the total weight of the composition.

These contents enable a deposit of make-up with good coverage on the lips.

“Pigments” shall mean mineral or organic, white or coloured particles, insoluble in an aqueous solution, intended for colouring and/or opacifying the resulting deposit. These can include mineral pigments, organic pigments and composite pigments (in other words pigments based on mineral and/or organic materials).

The “mineral pigments” include, for example, titanium dioxide; black, yellow, red and brown iron oxides and manganese violet.

The “organic pigments” can include, for example, pigments D & C red no. 19; D & C red no. 9; D & C Red no. 22; D & C Red no. 21; D & C Red no. 28; D & C Yellow no. 6; D & C orange no. 4; D & C orange no. 5; D & C Red no. 27; D & C red no. 13; D & C Red no. 7; D & C Red no. 6; D & C Yellow no. 5; D & C Red no. 36; D & C Red no. 33; D & C orange no. 10; D & C yellow no. 6; D & C Red no. 30; D &C red no. 3; D &C Blue 1; carbon black and cochineal carmine based lacquers.

### Additional Ingredients

The composition of the invention may further comprise a fatty pasty substance. Fatty pasty substance

The term “fatty pasty compound” or “pasty compound” or “fatty pasty substance” shall mean a non-crystalline fatty compound comprising, at a temperature of 25° C., a liquid fraction and a solid fraction.

The pasty compound is, for example, chosen from the group consisting of lanolin and its derivatives, silicone polymer compounds, alkyl methacrylate copolymers, preferably having a C8-C30 alkyl group, homo-oligomers and copolymers of vinyl esters having C8-C30 alkyl groups, homo-oligomers and copolymers of vinyl-ethers having C8-C30 alkyl groups, liposoluble polyethers resulting from the polyetherification between one or more C2-050 diols, ethylene-oxide and/or propylene-oxide copolymers with C6-C30 long-chain alkylene oxides, diglycerol esters, arachidyl propionate, phytosterol esters, non-cross-linked polyesters resulting from the polycondensation between a C4-C50 branched or linear carboxylic diacid or a polyacid and a diol or a polyol, the ester resulting from the esterification reaction of hydrogenated castor oil with isostearic acid such as a mono- di- or tri-isostearate of hydrogenated castor oil, a mixture of soya sterols and pentaerythritol oxyethylene (5 OE) oxypropylene (5 OP), fatty acid triglycerides and their derivatives, shea butter, cocoa butter, mango oil or butter, and the mixtures thereof.

According to a particular embodiment, the fatty pasty substance is shea butter or mango butter.

A composition according to the invention may have a content of pasty compounds ranging from 0.1% to 5%, for example from 0.2% to 4% by weight, preferably from 0.4 to 2% by weight, relative to the total weight of the composition.

### Fillers

The composition may further comprise at least one additional filler that is distinct from the silicone elastomer powder when it is present.

Within the meaning of the invention, the term “fillers” shall mean particles of any shape, colourless or white, mineral or organic, natural or synthetic, which are insoluble and dispersed in the medium of the composition. These fillers serve in particular to modify the rheology or the texture of the composition and/or provide a matt effect. The fillers can be mineral or organic and of any shape: platelets, spherical or oblong.

The “fillers” are chosen, in particular, from silicas, micas, of natural or synthetic origin, kaolin, zinc and titanium oxides; calcium carbonate, magnesium carbonate and bicarbonate; zinc, magnesium or lithium stearate, zinc laurate, magnesium myristate; synthetic polymer powders such as polyethylene, polyesters, polyamides (for example nylon); polyacrylic or polymethacrylic acid powders, mineral powders such as spherical silica; spherical titanium dioxides; glass and ceramic beads; organic material powders of natural origin such as starches from corn, wheat and rice, cross-linked or not, and the mixtures thereof.

According to a particular embodiment, silica, cellulose powder and the mixture thereof are used.

A composition according to the invention may have a content of filler or fillers ranging from 0.5% to 10%, for example from 1% to 8% by weight, preferably from 2% to 7% by weight, relative to the total weight of the composition.

### Other Colouring Materials

A composition for make-up of the lips can further comprise an additional “colouring material” that is distinct from the above-mentioned pigments and can be chosen from colouring materials that are water-soluble or insoluble, liposoluble or not, organic or inorganic, optical effect materials and the mixtures thereof.

According to a particular embodiment, the one on more additional colouring materials are chosen, in particular, from colouring agents, nacres and the mixtures thereof.

The term ‘colouring agents’ shall mean colouring agents that are conventionally used in the field of cosmetics, distinct from food colouring agents used in food products.

The “colouring agents” may include Yellow 5, Yellow 6, Blue 1, Green 5, Green 3, Green 6, Orange 4, Red 4, Red 21, Red 22, Red 27, Red 28, Red 33, Red 40, cochineal carmine (CI 15850, CI 75470). Liposoluble colouring agents are, for example, Sudan red, D&C Red 17, D&C Green 6, beta-carotene, Sudan brown, D&C Yellow 11, D&C Violet 2, D&C orange 5, quinoline yellow and annatto.

### Galenic

The composition of the invention is a make-up composition of keratin materials, in particular a lip make-up composition.

The composition of the invention is generally a solid composition such as previously defined.

Make-up compositions for the lips can include, in particular, a lipstick, a lip contour pencil or a lip balm.

According to a particular and preferred embodiment, it is a solid lip make-up composition, in particular a stick or lipstick.

### Cosmetic Method

The invention also relates to a method for cosmetic make-up of the lips, comprising the application of a composition according to the invention.

The composition applied on the lips is preferably a solid lip make-up composition, in particular a stick or lipstick.

The method according to the invention is, in particular, a method for make-up of the lips, intended to deposit on said lips a comfortable, matt, non-desiccating film, with good colour hold.

The invention will be illustrated by means of the following non-limiting examples. The percentages are expressed in percentage by weight relative to the total weight of the composition, unless otherwise indicated.

### EXAMPLES

### Example 1: Effect of the Presence of a Polar Wax in the Composition of the Invention

Two comparative formulas were tested, one with the presence of a polar wax (invention) and the other without the presence of polar wax (comparative). The polar wax used was candelilla wax.

The implementation of the said compositions was evaluated, as well as the stability and sensory properties of the composition.

The results are presented in the following table:

TABLE 1

| | | Composition |
|---|---|---|
| | Composition outside of | according to the |
| | the invention (without | invention (with |
| Ingredients | polar wax) | polar wax) |
| Silicone elastomer conveyed in an oil (DOWSIL EL | 25 | 25 |
| 9241 DM) | | |
| Silicone oil (DIMETHICONE 100 CS) | 7.8 | 7.8 |
| Lecithin EMULMETIK 300 IP | 0.5 | 0.5 |
| UNIPURE RED LC | 0.9875 | 0.9875 |
| C 339001 SUN PURO | 2.48 | 2.48 |
| UNIPURE RED LC | 0.77 | 0.77 |
| SUNCROMA FDC YELLOW 6 AL LAKE | 4.8 | 4.8 |
| SUNCROMA FDC YELLOW 5 AL LAKE | 2.6 | 2.6 |
| UNIPURE RED LC | 5.55 | 5.55 |
| Hydrogenated polydecene DEKANEX 2006 FG (non- | 28.0325 | 28.0325 |
| polar oil) | | |
| Polyethylene wax PERFORMALENE 500 (non-polar | 7.45 | 5.2 |
| wax) | | |
| Candelilla wax CANDELILLA WAX SR 3 (polar wax) | — | 2.5 |
| Silica SUNSPHERE H33 | 3 | 3 |
| Silica SILICA SHELLS | 2 | 2 |
| Silicone elastomer powder coated with silicone resin | 3 | 3 |
| (KSP-100) | | |
| Calcium carbonate | 0.05 | 0.05 |
| Shea butter | 1.5 | 1.5 |
| Stability | Presence of holes in | OK |
| | the sticks, impossible to | |
| | have a complete stick | |
| Sensory | Difficult to cast a correct | OK |
| properties | stick | |

The compositions are prepared according to the following protocol:
the silicone elastomer conveyed in an oil (Dowsil 9241 EL) and Dimethicone 100 cs are mixed under stirring at 95° C.; when the mixture is homogeneous, the polyethylene and candelilla waxes are incorporated;
the pigmentary paste comprising the pigments, hydrogenated polydecene and lecithin is added to the mixture prepared above when the waxes are melted, then the powders are sprinkled (fillers: silica shells, sunsphere H33 and KSP 100) under stirring;
the mixture at a temperature greater than 95° C. is poured into a mould.

The lipsticks are evaluated for several criteria:
Organoleptic properties: sensory properties, colour: the compositions undergo sensory testing for their properties of comfort, mattness and coverage.
Physicochemical properties: hardness, breakage test: the physicochemical measurements ensure the possible shaping of the composition of the invention, the strength of the stick during application and its breakage. In practice, the sticks are placed at 20° C. for 24h. They are then cut at 1 cm from the cup using a wire in order to measure the hardness of the stick. The operation is carried out three times in order to produce an average. The hardness makes it possible to predict the behaviour of the stick on release from the mould, on breaking and during application. A texture analyser is used for the hardness measurement, the TAXT Plus and the Exponent software. The value obtained, expressed in grams force (Gf), must be between 140 and 190.
Stability: The formulas are monitored in various ovens in order to reproduce the life of the product. These accelerated ageing tests make it possible to ensure that the product is stable over time. The durability of the product can also be confirmed under normal conditions of storage and use. In practice, the stability of the sticks is observed after passage through various ovens: humidity, alternation between 4° C. and 45° C. The parts are stabilised 24 hours after their manufacture. The samples are observed after a defined time in each oven, 24 hours, 2 weeks, 1 month, 3 months. The absence of colour drift is observed, as well as the absence of drops, exudation and changes in the appearance of the stick: glossiness, mattness.

The results show that the presence of the polar wax in the composition of the invention makes it possible to obtain a stable composition which has the desired organoleptic and physicochemical properties, compared with the same composition without polar wax.

This composition makes it possible to obtain a matt stick with high coverage, having a rather “rigid” structure in order to have an unguided and comfortable structure (no tightness over time). The term ‘unguided structure’ shall mean a formulation for application with the stick (lipstick tube) present outside of the mechanism

### Example 2: Velvety Matt Lipstick

TABLE 2

| Ingredients | % |
|---|---|
| Hydrogenated polydecene | QS 100 |
| Cross-linked silicone elastomer conveyed in an oil | 25.0 |
| (DIMETHICONE/DIMETHICONE CROSSPOLYMER - | |
| DOWSIL EL 9241 DM) | |
| Polyethylene wax | 5.5 |
| Candelilla wax | 3.0 |
| Shea butter | 1.5 |
| Pigments | 15.0 |
| Silica | 5.0 |

The composition is stable. After application on the lips, a comfortable deposit is obtained with good coverage, which is matt and non-desiccating, with a velvety appearance.

### Example 3: Matt Lipstick

TABLE 3

| Ingredients | % |
|---|---|
| Hydrogenated polydecene | QS 100 |
| Cross-linked silicone elastomer conveyed in | 25.0 |
| an oil (dimethicone/dimethicone | |
| crosspolymer DOWSIL EL 9241 DM) | |
| Dimethicone 100cs | 8.0 |
| Polyethylene wax | 4.2 |
| Cross-linked silicone elastomer powder | 2.5 |
| coated with silicone resin (vinyl | |
| dimethicone/methicone silsesquioxane | |
| crosspolymer, KSP-100) | |
| Candelilla wax | 2.5 |
| Shea butter | 1.5 |
| Pigments | 17.0 |
| Silica | 5.0 |

The composition is stable. After application on the lips, a comfortable deposit is obtained with good coverage, which is matt and non-desiccating, with a velvety appearance.

1-10. (canceled)

11. A cosmetic composition, comprising, in a physiologically acceptable medium, at least:
an organopolysiloxane elastomer conveyed in a first oil,
a non-polar oil distinct from the first oil,
a polar wax, and
pigments with a total content of greater than or equal to 10% by weight relative to the total weight of the composition.

12. The cosmetic composition according to claim 11, which is anhydrous.

13. The cosmetic composition according to claim 11, further comprising an organopolysiloxane elastomer powder distinct from the organopolysiloxane elastomer conveyed in the first oil.

14. The cosmetic composition according to claim 11, wherein the polar wax is selected from the group consisting of candelilla wax, beeswax, carnauba wax, rice bran wax, sunflower wax, jojoba wax and the mixtures thereof.

15. The cosmetic composition according to claim 14, wherein the polar wax is candelilla wax.

16. The cosmetic composition according to claim 11, further comprising at least one non-polar wax.

17. The cosmetic composition according to claim 11, wherein a total content of wax is greater than or equal to 5% by weight relative to the total weight of said composition.

18. The cosmetic composition according to claim 11, wherein the non-polar oil is selected from the group consisting of polybutylenes, hydrogenated polyisobutylenes, decene/butene copolymers, polybutene/polyisobutene copolymers, polydecenes, hydrogenated polydecenes and the mixtures thereof.

19. The cosmetic composition according to claim 11, wherein a content of non-polar oil ranges from 15% to 45% by weight relative to the total weight of said composition.

20. The cosmetic composition according to claim 11, wherein a total content of pigments ranges from 10% to 20% relative to the total weight of said composition.

21. The cosmetic composition according to claim 11, wherein the composition is a make-up product for the lips.

22. The cosmetic composition according to claim 21, wherein the composition is a lipstick, a lip contour pencil, or a lip balm.

23. A cosmetic method for make-up of the lips comprising applying, on the lips, at least one composition as defined in claim 11.
